(19) United States
(12) Patent Application Publication
(10) Pub. No.: US 2006/0165747 A1
(43) Pub. Date: Jul. 27, 2006

(54) Antifungal composition, method and kit for topically treating onychomycosis

(76) Inventor: David Rolf, Eden Prairie, MN (US)

Correspondence Address: James V. Harmon;US Bank Plaza, Suite 2000, 220 South Sixth Street, Minneapolis, MN, 55402, US

(21) Appl. No.: 11/040,725

(22) Filed: Jan. 24, 2005

(51) Int. Cl. A61K 31/496; A01N 25/00

(52) U.S. Cl. 424/405; 514/254.07

(57) ABSTRACT

An activator comprising a biocompatible base and a known fungicide in the form of an acid salt are mixed together by the user shortly before being applied to the infection so as to react them chemically to produce the free base of the fungicide thereby rendering the composition capable of penetrating throughout the infected tissue. The free base form has been found to be much more lethal to the fungal infection, particularly in the treatment of onychomycosis. The activator is formulated to be mixed within a few days of or just before application with the fungicide, e.g., terbinafine hydrochloride (an acid salt). Although the free base is subject to degradation and has a shorter shelf life, it was found that mixing just prior to use eliminates problems that could be caused by degradation of an active ingredient. A preferred activator solution also contains a penetration enhancer and a tissue softener.

### FIELD OF THE INVENTION

This invention relates to medical therapy and more particularly to the treatment of fungal infections.

### BACKGROUND OF THE INVENTION

It is estimated that perhaps 30,000,000 Americans have onychomycosis, fungal infections of the fingernails or toenails including the nail matrix, bed or plate. The primary protein constituent of human nails, hair, and stratum corneum is keratin, however with respect to chemical composition and permeability, human nails are more like hair then stratum corneum. Nitrogen is a major component of the nail since it is primarily proteinaceous. The total lipid content of a mature nail is less then 1% while the skin stratum corneum can contain an order of magnitude more lipid. The level of hydration of the nail is typically much lower then the stratum corneum. The nail plate is 100-200 times thicker then stratum corneum and has a high affinity for binding and retaining many drugs. These are some reasons that topical nail therapy has generally been ineffective.

The most effective FDA approved medical treatment for these infections are oral antifungals such as terbinafine HCl or griseofulvin. These oral medications suffer various significant shortcomings. Treatment is expensive; the prescription can be as much as $600 for one course of treatment. Relapse or reinfection rates can be as high as 30% after successful treatment. These systemic medications also typically possess significant interactions with other medications, alcohol, and have a potential for serious liver toxicity.

The disadvantage of systemic drug administration to treat localized peripheral conditions such as nail disease is clear. The need for an effective topical or local treatment for onychomycosis through the transungual route (through the nail bed) is obvious, yet no effective transungual treatment has been developed despite the existence of numerous fungicidal medications and formulations. The difficulty of delivering a therapeutic dose of antifungal to the site of infection seems great indeed.

Some fungal infections involved in onychomycosis include Epidermophyton, Microsporum, Trichophyton, Candida Albicans, and Trichophyton Rubrum. There are many pharmaceutical agents with antifungal activity including the following classes: azoles such as ketoconazole, miconazole, bifonazole, butoconazole, clotrimazole, croconazole, eberconazole, econazole, oxiconazole, fenticonazole, iosconazole, sulconazole and triconazole, lanoconazole, neticonazole, omoconazole, setraconazole; allylamines, such as terbinafine and natrifine; morpholines such as amorpholine; polyenes, such as amphoteracin B, nystatin, and natamaycin.

Other antifungal agents include; flucytosine, griseofulvin, potassium iodide, butenafine ciclopirox, ciloquinol (iodochlorhydroxyquin), haloprogin, tolnaftate, aluminum chloride 30%, Catellant's paint, undecylenic acid compound, gentian violet, oil of bitter orange, potassium permanganate, propylene glycol 50% selenium sulfide (2.5% lotion), Whitfield's ointment, zinc pyruthione, betulin and its derivatives, and some essential oils like tea tree oil, eucalyptus, etc.

The nails have always represented a formidable permeation barrier to anyone attempting to deliver a fungicidal drug to the base of the base of the nail bed topically (transungually). Numerous approaches to this problem are documented in the patent art including numerous polymeric compositions containing drugs, which are painted onto the nails (U.S. Pat. No. 6,319,509, U.S. Pat. No. 6,214,360; WO 8,702,580; U.S. Pat. No. 5,264,206.

Numerous patents demonstrate the utility of keratin softening agents such as urea and antioxidants (U.S. Pat. No. 6,743,417 and U.S. Pat. No. 6,673,842), triacetin, lauramide diethanolamine, caproic triglyceride, cineol, and/or sulfhydral containing amino acids (U.S. Pat. No. 6,585,963) to help swell the keratin structure of the nail thereby increasing its permeability to antifungal agents. Other patents (U.S. Pat. No. 6,455,592, U.S. Pat. No. 5,814,305,) describe the utility of hydrophilic penetration agents such as glycols, glycol monoethers, glycol diethers, dimethyl sulfoxide, caprolactam, dimethylisosorbide, iospropylidene glycerol, dimethylimidazolidinone, ethyl lactate, polyoxyethylenated C8-C10 glycerides, polyethylene glycol 20 glycerol laurate, dimethylacetamide, isopropanol, n-butanol, ethanol, dipropyleneglycol monoethylether, and others. All these compositions are claimed to work with the hydrochloride salt of terbinafine or an antifungal active ingredient. Nevertheless, current topical preparations are not capable of penetrating throughout the infected area. In addition to efficacy, a successful product must also have an adequate shelf life. It is not practical to provide a commercial pharmaceutical preparation with a shelf-life significantly less then 2 years since it can take up to six months from manufacturing date to get the product on the store shelves.

### SUMMARY OF THE INVENTION

The present invention provides a method and an activator composition that is added to a known fungicide which, after following instructions, will either eradicate or greatly improve fungal infections of the foot, particularly onychomycosis. The invention also utilizes a kit comprising an activator for a known fungicidal preparation that when mixed together by the user shortly before being applied react and thereby render the composition capable of penetrating throughout the infected tissue. When mixed, the activator reacts chemically with the fungicide which is an acid salt converting it to the free base; a form that I have found to be much more lethal to the fungal infection, particularly in the treatment of onychomycosis. The activator is formulated to be mixed within a few days of or just before application with a fungicide such as LAMISIL® which contains terbinafine hydrochloride (an acid salt) as the active ingredient. A patient who is motivated to be free from onychomycosis infection can easily prepare the mixture before it is applied. Although the free base is subject to degradation and has a shorter shelf life, it was found that mixing just prior to use eliminates problems associated with degradation of an active ingredient before use.

Alternatively, terbinafine tablets are used as an active fungicide in place of LAMISIL®. After crushing into a powder, the terbinafine hydrochloride is dissolved in a minimal amount of water, reacted with the activator to release the free base of the fungicide and dissolved in a solvent, e.g., isopropyl alcohol, most preferably with other penetration enhancing substances. Other fungicidal preparations can be used with the activator if desired.

### DETAILED DESCRIPTION OF THE INVENTION

In accordance with the invention, the activator solution is used to convert a fungicidal acid salt to the free base form. In this form, the fungicide becomes capable of penetrating throughout the infected tissue. For any particular fungicide and dosage form (liquid, cream, ointment, tablet, etc.) one or a combination of activator reagents can be added drop-wise, for example, as a simple means of volumetric measurement. The activator solution of the present invention provides the following benefits:

1. It reacts with the acid salt of the fungicide to release the free base which has a much greater fungicidal activity than an acid salt form.

2. It dissolves the free base and keeps it in solution

3. It optionally provides a nail penetration enhancer(s).

4. It optionally adds a nail-softening agent, which helps erode the diseased nail and improves penetration of the fungicide.

The invention can be provided as a kit that includes a container, the activator solution and instructions for use. In one form of kit which accomplishes the goals noted above while avoiding potential stability problems of the free base form of the anti-fungal compound, a two-component ampoule contains the stable salt (solution or solid) form of the antifungal agent in one compartment and the penetration enhancing activator solution in the other compartment. The barrier between these compartments is broken and the materials are then mixed just before application. A kit of this kind enhances convenience, portability and maintains stability of the preparation.

To use the product, a patient prepares the mixture of the activator solution and fungicide, then applies the solution to his or her nails and generally to the surrounding tissue daily using an eyedropper. The viscosity and surface tension of the liquid preparation is preferably low enough so that the liquid flows readily across and into the interstitial spaces of the nail and surrounding region. Depending on the refractory nature of the infection and the rate of growth of the nail, complete treatment could require up to six months or more. Once nails have returned to their normal appearance with no evidence of fungal infection, continued application of the antifungal solution, most preferably without the nail softener, will help assure against reinfection or relapse and help provide a healthy strong nail.

A typical kit for converting terbinafine hydrochloride (LAMISIL®) or other fungicide into an onychomycosis treatment solution comprises the following:

1. The penetration enhancing activator solution in a container.

2. Instructions for mixing the activator with a fungicide to form the free base of the fungicide and for applying the solution to the nails and surrounding skin.

3. A vial to spray LAMISIL® or other fungicide into the activator solution to react therewith.

4. A disposable eyedropper to apply the reacted composition to the skin and/or nail area.

It has been found that the free base of the fungicide penetrates nail material more effectively than the acid salt form which is always water-soluble. This phenomenon also holds true for skin permeation where under passive diffusion, acid salts were found to penetrate the stratum corneum (chief barrier layer of the skin) less readily than uncharged equivalent molecules.

The base for neutralizing the acid salt does not need to be a strong base. Many different bases will suffice, however they should be soluble in the solution, non-irritating to the skin, reasonably stable and non-sensitizing. Some preferred examples include weak inorganic bases such as alkali metal or alkaline earth metal carbonates, bicarbonates, phosphates or hydroxides. Organic bases such as di- or triethanolamine, and other non-volatile amines are suitable. Organic acid salts such as citrate, tartarate, oxalate, etc., will also work nicely.

Typical solubilizers for these preparations are water miscible low molecular weight alcohols, like methanol, ethanol, isopropanol, butanol or low molecular weight glycols like propylene glycol, 1,3 propanediol, butanediols or butanetriols, polyols or low molecular weight carbonate esters, lactones, lactams, esters or amides. Of course these solubilizers must be non-toxic, non-irritating, non-sensitizing and biocompatible. They must not react with the fungicide to alter or degrade it and must be generally stable enough to work with. Volatile solubilizers have the added advantage of creating a higher concentration of the fungicide at the application site after they evaporate. This enhances absorption/permeation of the fungicide into the nail. Ideally, the fungicide should be at or near a solution saturated state after application to maximize absorption.

The primary function of the solubilizer or solvent contained in the activator solution is to dissolve the free base of the drug in a minimal amount of solution since every milliliter of activator added dilutes the antifungal agent and lowers the solution's potency. Another function of the solvent is to dissolve the nail-softening component of the preparation.

Typical softeners employed in accordance with the invention comprise keratin plasticizers or reducing agents that break up the inter-chain hydrogen bonding in the nail keratin protein. Examples include urea, low molecular weight amides, carbamides, lactams, and amines. Other nail softeners break disulfide cross linkages in the keratin by reducing disulfides to sulfhydrals with agents such as non-volatile low molecular weight organic thiols like cysteine, N-acetyl-L-cysteine, thoisugars, thio-glycols, mixed thio-alcohols, organic or inorganic weak reducing agents, etc. Of course the nail softener must be non-irritating, biocompatible, and non-sensitizing.

The penetration-enhancing component of the solution is a polar organic solvent, e.g. a polar aprotic solvent that is miscible in the mixture. The penetration-enhancer should be polar enough to permeate and swell nail and skin keratin and also dissolve the antifungal agent. It should not react with the drug and should flow well over and into the application site. Some typical penetration enhancers include glycols, glycol monoethyl ethers, glycol diethers, dimethyl sulfoxide DMSO, dimethylforamide DMF, decanemethyl sulfoxide C10 MSO, caprolactam, dimethylisosorbide, methyl or ethyl carbonates, methyl or ethyl carbamates, dipropylene glycol dimethyl ether, iospropylidene glycerol, dimethyl imidazolidinone, methyl or ethyl lactate, ammonium lactate, polyoxyethylenated C8-C 10 glycerides, polyethyleneglycol 20 glycerol laurate, dimethylacetamide.

Typical activator solutions, e.g., as set forth below, are added drop-wise to 500 mg of LAMISIL® spray for athlete's foot to make a solution for treating onychomycosis.

| Activator: any of the following: | |
|---|---|
| NaHCO3, Na2CO3, KHCO3 K2CO3, triethanolamine | 0.02-0.1% |
| Solubilizer: any of the following: | |
| Iospropanol, ethanol, methanol, propylene glycol | 20-40% |
| Pentreaton enchancer: any of the following: | |
| DMSO, DMF, DMA, dimethylacetamide | 10-30% |
| Transcutol | 20-50% |
| Softener: either of the following: | |
| Urea, cysteine | 1-15% |

All quantities herein are expressed as parts or percent by weight unless otherwise stated.

In one embodiment, a two-part topical liquid dosage package includes the fungicide, e.g., Terbinafine HCl in one compartment of an ampoule and the activator solution in a separate compartment. Just before use, a barrier between the compartments is ruptured, allowing the two liquids to be mixed together. The newly mixed solution is then applied to the infected nails and surrounding skin. Each dose is a single dose quantity mixed freshly before use. One suitable two-part ampoule dosage container is available from James Alexander Corporation of Blairstown, N.J.

If desired, the patient can presoak the infected nails or other tissue in warm water, a warm soap solution or use the invention after bathing or swimming to further facilitate absorption of the compositions described herein. The activator solution of the invention will be better understood by reference to the following examples:

| | Example 1 | Example 2 |
|---|---|---|
| Na2CO3 (sodium carbonate) | 0.02% | 0.05% |
| Ethanol | 30% | 25% |
| DMF (dimethyl formamide) | 30% | 25% |
| Transcutol (deithylene glycol monoethylether) | 30% | 25% |
| Cysteine | 9.98% | 24.95% |
| | Example 3 | Example 4 |
| KHCO3 (potassium bicarbonate) | 0.02% | 0.08% |
| Methanol | 30% | 25% |
| DMA (diethylene glycol monoethylether) | 30% | 25% |
| Transcutol (deithylene glycol monoethylether) | 30% | 25% |
| Urea | 9.98% | 24.92% |
| | Example 5 | Example 6 |
| KHCO3 (potassium bicarbonate) | 0.02% | 0.05% |
| Propylene glycol | 30% | 25% |
| DMSO (dimethyl sulfoxide) | 30% | 25% |
| Transcutol (deithylene glycol monoethylether) | 30% | 25% |
| Urea | 9.98% | 24.95% |
| | Example 7 | Example 8 |
| Triethanolamine | 0.04% | 0.08% |
| Isopropanol | 40% | 25% |
| DMSO (dimethyl sulfoxide) | 25% | 25% |
| Transcutol (deithylene glycol monoethylether) | 34.96% | 25% |
| Urea | 0% | 24.92% |

| | Example 9 |
|---|---|
| Triethanolamine | 0.04% |
| Isopropanol | 30% |
| DMSO (dimethyl sulfoxide) | 30% |
| Transcutol (deithylene glycol monoethylether) | 30% |
| Cysteine | 9.96% |

Procedure and Results in Treating a Human Patient

The present invention was employed in treating chronic fungal infection of a patient which involved the great toe nail on both feet that had been infected with trichophytan rubum fungus for many years prior to being treated with the present invention LAMISIL® was used as a fungicide with an activator solution as described above in examples 5, 7, and 9. Approximately 0.25-0.5 ml of LAMISIL® was sprayed into a test tube and an approximately equal volume of activator solution was added drop-wise and mixed with the fungicide solution just prior to application. Any leftover solution was used for the next application of treatment solution. An eyedropper was used to apply the improved antifungal penetration liquid to the tops of nails and on all tissues surrounding the nails. The low viscosity of the solution helped it to flow into all corners and cracks of the nails. The solution was applied about every 2 or 3 days and photographic evaluation was performed by estimating the percent of white colored infected region for each nail. Results indicated that after 11 weeks the fungal infection decreased as shown in the table below.

| | Left Great Toe | Right Great Toe |
|---|---|---|
| Initial | 74% infected | 71% infected |
| Eleven weeks | 29% infected | 20% infected |

Each toe was photographed and the white region indicated the area of infection. Copies of the photos were made and the percent infected calculated by cutting and weighing the white portions (infected) of each photo divided by the whole toenail area and expressing the result as a percent.

Many variations of the present invention within the scope of the appended claims will be apparent to those skilled in the art once the principles described herein are understood.

What is claimed is:

1. A fungicide intensifier composition to render a fungicide more lethal in the destruction of a fungal infection, said composition comprising, a polar liquid solubilizer and an activator dissolved in the solubilizer, said activator comprising a biocompatible base that reacts chemically with an acid salt of a fungicide to form the free base thereof when mixed therewith for use in applying the freebase of the fungicide topically to the fungal infection so as to reduce or eliminate one or more of the symptoms of the fungal infection.

2. The composition of claim 1 wherein the solubilizer comprises at least one member selected from the group consisting of water, isopropanol ethanol, methanol, propylene glycol, ethylene glycol, methylene glycol, butanol, butylene glycol 1, 3 propanediol, butanediol, butanetriol, a polyol, a low molecular weight carbonate ester, a lactone ester, a lactam, and an amide.

3. The composition of claim 1 wherein the fungal infection comprises onychomycosis and the activator comprises at least one member selected from the group consisting of the basic salt of an alkali metal, the basic salt of an alkaline earth metal, an organic acid salt and a basic amine.

4. The composition of claim 1 wherein the solubilizer comprises at least one member selected from the group consisting of isopropanol, ethanol, methanol, propylene glycol, butanediols, butanetriols, ethylene glycol, methylene glycol, butanol, butalene glycol, 1,3 propanediol, a polyol, a low molecular weight carbonate ester, a lactone ester, a lactam, an amide and water, the fungal infection comprises onychomycosis and the activator comprises at least one member from the group consisting of the basic salt of an alkali metal, the basic salt of an alkaline earth metal, an organic acid salt and a basic amine.

5. The composition of claim 1 wherein the solubilizer comprises at least one member selected from the group consisting of isopropanol, ethanol, methanol, propylene glycol, ethylene glycol, methylene glycol, butanol, butalene glycol, 1, 3 propanediol, a polyol, a low molecular weight carbonate ester, a lactone ester, a lactam, an amide and water, the fungal infection comprises onychomycosis and the activator comprises at least one member selected from the group consisting of the basic salt of an alkali metal, the basic salt of an alkaline earth metal, organic acid salt, an organic acid salt and a basic amine, and including a tissue penetration enhancer comprising a polar organic solvent and a tissue softener comprising a biocompatible agent for breaking inter-chain hydrogen bonding of keratin molecules to thereby render the tissue more flexible.

6. The composition of claim 1 including a penetration-enhancing agent comprising a biocompatible polar organic solvent dispersed in said solution.

7. The composition of claim 1 including a softener for breaking chemical bonds within the nail and stratum corneum to render the tissue more pliable.

8. The composition of claim 1 including a biocompatible tissue softener for rendering the infected tissue more pliable to thereby enhance penetration of the solution into fungus infected tissue.

9. A fungicide intensifier kit to render a fungicide more lethal in the destruction of an onychomycosis infection, said kit including an activator comprising a biocompatible base for entering into a chemical reaction with an acid salt of a fungicide to form the free base thereof, a container for the activator, instructions for mixing the activator with the fungicide and for applying the resulting free base of the fungicide to the nails and/or surrounding skin, and an applicator to apply the mixture of the activator and the fungicide to the infected tissue so as to reduce or eliminate one or more of the symptoms of the fungal infection.

10. The kit of claim 9 including a penetration enhancer comprising a biocompatible polar organic solvent and a tissue softener comprising a biocompatible keratin plasticizer or reducing agent for breaking inter-chain hydrogen bonds between keratin molecules to render the tissue more flexible.

11. The kit of claim 9 wherein the activator comprises at least one member selected from the group consisting of a basic salt of an alkali metal, a basic salt of an alkaline earth metal, an organic acid salt and an amine.

12. The kit of claim 10 wherein the penetration enhancer comprises a member selected from the group consisting of DMSO, DMF, dimethyl acetamide DMA and transcutol.

13. The kit of claim 10 wherein the tissue softener comprises urea or cysteine.

14. A method of treating a fungal infection wherein a fungicide is rendered more lethal in the destruction of a fungal infection, said method comprising, providing an activator comprising a biocompatible base adapted to enter into a chemical reaction with a fungicide, providing the acid salt of fungicide, placing the activator in solution with the acid salt of the fungicide to thereby react the activator with the fungicide to form the free base thereof and applying the resulting solution containing the free base of the fungicide to the infected tissue to thereby eliminate or reduce one or more of the symptoms of the fungal infection.

15. The method of claim 13 including the step providing as said activator a biocompatible basic salt of an alkali metal, an alkaline earth metal basic salt, an organic acid salt or an amine and providing as said fungicide at least one member selected from the group consisting of an azole, ketoconazole, miconazole, bifonazole, butoconazole, clotrimazole, croconazole, eberconazole, econazole, oxiconazole, fenticonazole, iosconazole, sulconazole, triconazole, lanoconazole, neticonazole, omoconazole, setraconazole, allylamines, terbinafine, natrifine, morpholines, amorpholine, polyenes, amphoteracin B, nystatin and natamaycin, flucytosine, griseofulvin, potassium iodide, butenafine ciclopirox, ciloquinol (iodochlorhydroxyquin), haloprogin, tolnaftate, aluminum chloride, Catellant's paint, undecylenic acid compound, gentian violet, oil of bitter orange, potassium permanganate, propylene glycol 50% selenium sulfide (2.5% lotion), Whitfield's ointment, zinc pyruthione, betulin and its derivatives, an essential oil of tea tree and eucalyptus oil.
